(19) United States
(12) Patent Application Publication
(10) Pub. No.: US 2009/0010902 A1
(43) Pub. Date: Jan. 8, 2009

(54) TLR4 Transcription Activity Suppressor

(75) Inventor: Takashi Masuda, Tokyo (JP)

Correspondence Address: EDELL, SHAPIRO & FINNAN, LLC, 1901 RESEARCH BLVD., SUITE 400, ROCKVILLE, MD, 20850-3164, US

(73) Assignee: Toa Pharmaceutical Co., Ltd., Tokyo (JP)

(21) Appl. No.: 11/774,123

(22) Filed: Jul. 6, 2007

(51) Int. Cl. A61K 45/00; A61P 1/04

(52) U.S. Cl. 424/93.41

(57) ABSTRACT

The object of the invention is to provide a TLR4 transcription factor activity suppressor which suppresses TLR4 transcription factor and is effective in inducing or maintaining remission of ulcerative colitis.

A TLR4 transcription factor activity suppressor comprising butyric acid bacteria, a metabolite of butyric acid producing bacteria, a metabolite of butyric acid bacteria, or butyric acid is provided.

### BACKGROUND OF THE INVENTION

1. Field of the Invention

The present invention relates to a TLR4 transcription factor activity suppressor effective in inducing remission or maintaining remission of ulcerative colitis. The present invention also relates to a remission inducing agent or remission maintaining agent for ulcerative colitis.

2. Description of the Related Art

Toll-like receptor (TLR) is a receptor which is the first to act in natural immunity. Ten types of TLR have been discovered. These are molecules which recognize the various molecular structures of PAMP (pathogen-associated molecular patterns) which are common to pathogens. PAMP is the pathogen molecular structure which can be recognized by innate immunity cells.

With the PAMP recognition by TLR, the immune reaction by phagocytosis cells and lymphocytes is induced. As a result, control of TLR can lead to control of the entire immune reaction.

One of the TLRs is TLR4 which is known to recognize the gram negative bacterial cell wall (lipopolysaccharide, LPS) In particular, it has been reported that the expression amount of TLR4 is higher inside the intestinal tract of ulcerative colitis patients as compared to that of normal people.

Ulcerative colitis (UC) is a disease which has been designated as intractable to treatment, and as of yet there is no standard of treatment. Currently, remission is maintained by 5-ASA medicines such as mesalazine, salazosulfapyridine, and the like, or inflammation is reduced by steroids. The action mechanism for UC patients is unknown. In addition, there have been no reports what effect they have on the TLR expression amount.

Therefore, if the overexpression of TLR4 is reduced in UC patients, it is thought that this will reduce inflammation reactions. Currently, the transcription control of TLR4 is not known.

### SUMMARY OF THE INVENTION

The problem to be solved by the present invention is to provide a TLR4 transcription factor activity suppressor which suppresses TLR4 transcription factor and is effective in inducing remission and maintaining remission of ulcerative colitis.

After intensive study, the present inventors found that the culture supernatant of butyric acid bacteria reduced the TLR4 mRNA expression. In addition, butyric acid, which is one of the metabolic products contained in the culture supernatant of the butyric acid bacteria, similarly reduced mRNA expression of TLR4. Based on these findings, the present invention was completed. Furthermore, the butyric acid bacteria culture supernatant and butyric acid was found not only to decrease the expression amount of TLR4 mRNA but also reduced the expression amount of TLR4 protein.

In other words, the present invention relates to

1. A TLR4 transcription factor activity suppressor comprising butyric acid bacteria, a metabolite of butyric acid bacteria, a butyric acid bacteria formulation or butyric acid.
2. A TLR4 transcription factor activity suppressor as described in 1, wherein said metabolite of butyric acid bacteria is a culture supernatant of butyric acid bacteria or a supernatant of a mixed culture of butyric acid bacteria, lactic acid bacteria, and saccharifying bacteria.
3. A TLR4 transcription factor activity suppressor as described in 1, wherein said butyric acid bacteria formulation is a formulation of a mixture of butyric acid bacteria, lactic acid bacteria, and saccharifying bacteria.
4. A remission inducing agent or remission maintaining agent for ulcerative colitis comprising butyric acid bacteria, a metabolite of butyric acid bacteria, a butyric acid bacteria formulation, or butyric acid.
5. A remission inducing agent or remission maintaining agent for ulcerative colitis as described in 4, wherein said butyric acid bacteria is included in a formulation of a mixture of butyric acid bacteria, lactic acid bacteria, and saccharifying bacteria.
6. A remission inducing agent or remission maintaining agent for ulcerative colitis as described in 4, wherein said metabolite of butyric acid bacteria is a culture supernatant of butyric acid bacteria or is a supernatant of a mixed culture of butyric acid bacteria, lactic acid bacteria, and saccharifying bacteria.

By oral administration of the TLR4 transcription factor activity suppressor of the present invention, TLR4 expression is suppressed. With this, remission of ulcerative colitis is induced or maintained.

### BRIEF DESCRIPTION OF THE DRAWINGS

FIG. 1 shows an effect on TLR4 mRNA of various bacterial culture supernatant addition to human intestinal epithelial cells strain HT-29.

FIG. 2 shows a change in UC-DAI.

### DESCRIPTION OF THE PREFERRED EMBODIMENTS

The present invention will be described in detail below.

The TLR4 transcription factor activity suppressor of the present invention contains butyric acid bacteria, a metabolite of butyric acid bacteria, a butyric acid bacteria formulation, or butyric acid.

Butyric acid bacteria of the present invention is Clostridium butyricum which produces n-butyric acid.

For the mixture formulation of butyric acid bacteria, lactic acid bacteria, and saccharifying bacteria, the butyric acid bacteria, lactic acid bacteria, and saccharifying bacteria are cultured, and after completing culturing, the bacteria are collected by centrifugation, and this is mixed with a stabilizer and freeze dried. After drying, this is pulverized and mixed with a suitable base substance such as cornstarch, potato starch, dextrin, and the like. This formulation is constituted from butyric acid bacteria alone or from butyric acid bacteria, lactic acid bacteria, and saccharifying bacteria.

The butyric acid metabolic product of the present invention is the metabolite obtained from culturing butyric acid bacteria. The metabolite of the present invention can be the culture supernatant obtained after culturing butyric acid bacteria. After further drying the culture supernatant, this is mixed with a suitable base material such as cornstarch, potato starch, dextrin and the like. For the metabolic product of butyric acid bacteria, the supernatant of a mix culture of butyric acid bacteria, lactic acid bacteria, and saccharifying bacteria can also be used.

The TLR4 transcription factor activity suppressor of the present invention can be butyric acid live bacteria by itself or it can be a mixture of butyric acid bacteria, lactic acid bacteria, and saccharifying bacteria. It is preferably administered in the form of powder, fine granule, granule, tablet, or the like. The present invention can be butyric acid bacteria culture supernatant powder by itself, or it can be a supernatant powder of a culture of butyric acid bacteria, lactic acid bacteria, and saccharifying bacteria. This can be administered as powder, fine granule, granule, tablet or the like.

The TLR4 transcription factor activity suppressor of the present invention can be butyric acid bacteria or dried bacteria of butyric acid bacteria, lactic acid bacteria, and saccharifying bacteria. When this is a powder, fine granule, granule, or tablet, the concentration range should be from 100 mg to 10 g. If it is a powder of a culture supernatant obtained after culturing butyric acid bacteria, lactic acid bacteria, and saccharifying bacteria, the concentration range should be 0.1 mg to 1000 mg.

The TLR4 transcription factor activity suppressor of the present invention suppresses the TLR4 expression. With this, it is anticipated that remission of ulcerative colitis is induced or maintained.

### Embodiment 1

Each of Clostridium butyricum TO-A strain (abbreviated as “CB”), Streptococcus faecalis (abbreviated as “SF”), and Bacillus mesentericus (abbreviated as “BM”) is inoculated onto PYG modified medium. This was cultured for 24 hours at 37 degrees C. After culturing, this was centrifuged, and the supernatant was extracted. The supernatant was sterilized by filtration and used.

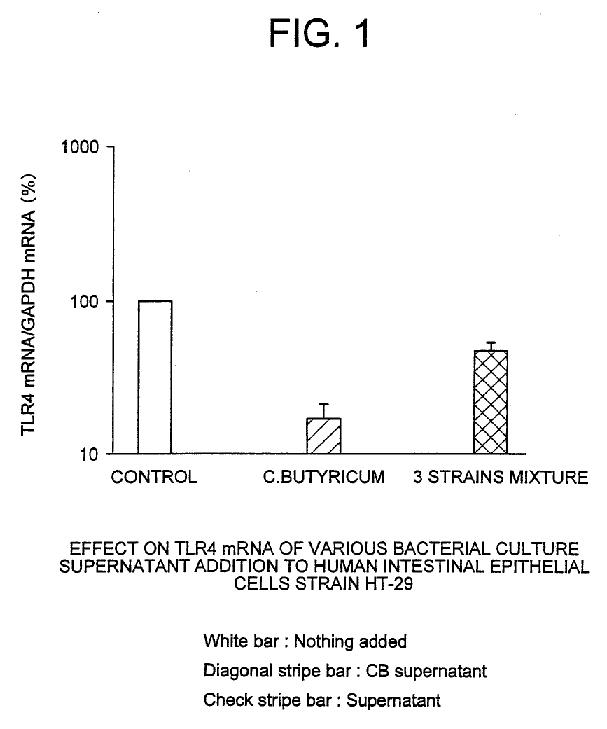

The CB culture supernatant or a mix supernatant of three bacteria types of CB, SF, and BM was added to human intestinal tract epithelial cell strain HT-29. Total RNA was extracted from HT-29, and TLR4 mRNA expression amount was measured by real time RT-PCR. With the non-stimulation TLR4 mRNA/GAPDH mRNA as 100%, the results are shown in FIG. 1.

By adding CB culture supernatant and supernatant of the 3 types of bacteria, the TLR4mRNA expression amount was dramatically reduced to 16±8%.

### Embodiment 2

With respect to 10 ulcerative colitis patients with mild or medium symptoms, a mixture formulation of butyric acid bacteria, lactic acid bacteria, and saccharifying bacteria was administered for 4 weeks. The clinical status, colonoscopy finding, UC-DAI (ulcerative colitis disease activity index), and the like were studied. UC-DAI is a numerical value showing the severity of ulcerative colitis. It takes into account the number of bowel movements, presence or absence of blood in the stool, colonoscopy finding, overall evaluation, the patients own impression, and the like. Higher UC-DAI number represents more severe disease. The patients ranged in age from 18 to 54 years old. There were 5 males and 5 females. Prior to administration, the number of bowel movements per day ranged from 2 to 8 times. Of 6 cases, 5 cases had blood in the stool. Four cases had abdominal pain. The formulation of the mixture of butyric acid bacteria, lactic acid bacteria, and saccharifying bacteria was taken daily until after the end of the trial.

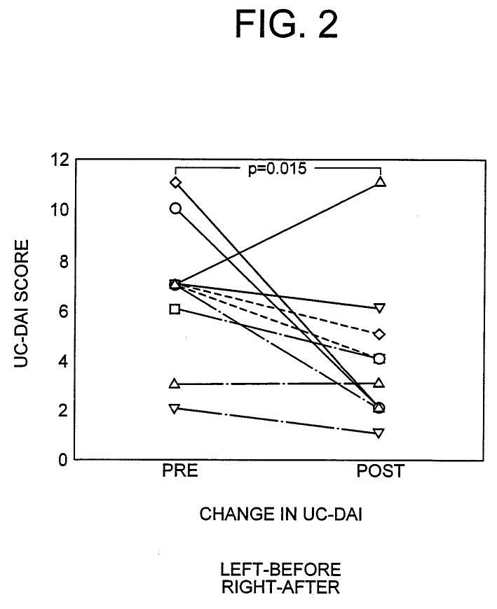

After administration of the formulation, with the colonoscopy findings, almost all of the cases had milder or improved inflammation findings. As shown in FIG. 2, in comparing the UC-DAI prior to administration (8.00±1.83), there was a significant improvement after administration to a score of 3.83±1.46 (p=0.015).

With the TLR4 transcription factor activity suppressor of the present invention, remission-inducing or remission maintaining effect for ulcerative colitis is anticipated. This will contribute greatly to the treatment of ulcerative colitis.

What is claimed is:

1. A TLR4 transcription factor activity suppressor comprising butyric acid bacteria, a metabolite of butyric acid bacteria, a butyric acid bacteria formulation or butyric acid.

2. A TLR4 transcription factor activity suppressor as described in claim 1, wherein said metabolite of butyric acid bacteria is a culture supernatant of butyric acid bacteria or a supernatant of a mixed culture of butyric acid bacteria, lactic acid bacteria, and saccharifying bacteria.

3. A TLR4 transcription factor activity suppressor as described in claim 1, wherein said butyric acid bacteria formulation is a formulation of a mixture of butyric acid bacteria, lactic acid bacteria, and saccharifying bacteria.

4. A remission inducing agent or remission maintaining agent for ulcerative colitis comprising butyric acid bacteria, a metabolite of butyric acid bacteria, a butyric acid bacteria formulation, or butyric acid.

5. A remission inducing agent or remission maintaining agent for ulcerative colitis as described in claim 4, wherein said butyric acid bacteria formulation is a formulation of a mixture of butyric acid bacteria, lactic acid bacteria, and saccharifying bacteria.

6. A remission inducing agent or remission maintaining agent for ulcerative colitis as described in claim 4, wherein said metabolite of butyric acid bacteria is a culture supernatant of butyric acid bacteria or is a supernatant of a mixed culture of butyric acid bacteria, lactic acid bacteria, and saccharifying bacteria.
